(19) United States
(12) Patent Application Publication
(10) Pub. No.: US 2022/0039754 A1
(43) Pub. Date: Feb. 10, 2022

(54) ELECTRONIC DEVICE FOR RECOMMENDING CONTENTS

(71) Applicant: SAMSUNG ELECTRONICS CO., LTD., Suwon-si (KR)

(72) Inventors: Hyungi AHN, Suwon-si (KR); Donghyun ROH, Suwon-si (KR); Kyungsub MIN, Suwon-si (KR)

(21) Appl. No.: 17/391,365

(22) Filed: Aug. 2, 2021

(30) Foreign Application Priority Data
Aug. 10, 2020 (KR) 10-2020-0099754

(51) Int. Cl. A61B 5/00; G06N 5/04; G06F 17/16; G06K 9/62

(52) U.S. Cl. CPC A61B 5/7264; A61B 5/7246; G06K 9/6263; G06N 5/04; G06F 17/16; A61B 5/7221

(57) ABSTRACT

An electronic device in various embodiments may include: a communication circuit configured to communicate with an external electronic device; a memory configured to store user reaction data indicating reactions to contents for users and biostatistic data for users; and a processor connected to the communication circuit and the memory, wherein the processor is configured to: calculate preference values for contents, to which users have reacted, for users using the user reaction data; predict preference values for contents, which have not been exposed to users, for users using the calculated preference values; generate profiles related to states or activities for users using the biostatistic data; calculate reliability values for the generated profiles; calculate relevance values indicating degrees of relevance between profile types and contents based on the calculated preference values, the predicted preference values, and the calculated reliability values; select, from among contents which have not been exposed to a user selected from among the users, a content to be recommended to the selected user based on the predicted preference values; select, as a profile for generation of a recommendation reason, at least one of profiles of the selected user based on reliability values for the profiles of the selected user and relevance values between the recommended content and the profile types; and control the communication circuit to transmit the selected profile and the recommended content to an external electronic device of the selected user.

### CROSS-REFERENCE TO RELATED APPLICATION

This application is based on and claims priority under 35 U.S.C. § 119 to Korean Patent Application No. 10-2020-0099754, filed on Aug. 10, 2020, in the Korean Intellectual Property Office, the disclosure of which is incorporated by reference herein in its entirety.

### BACKGROUND

### Field

The disclosure relates to an electronic device configured to recommend, to a user, contents related to the user's taste.

### Description of Related Art

An electronic device may collect raw data indicating a user's reaction (e.g., ratings indicating “helpful” or “not helpful”, viewing time, or whether content has been purchased) to a content, and may predict the user's reaction to a content that the user has not yet experienced, based on the collected raw data. This prediction technique may be referred to as matrix completion, and matrix factorization may be used as a representative prediction technique. An example of using matrix factorization is as follows.

A level (preference) at which a user likes a content may be expressed as an element (entry), r_ui, of an R(U×I) matrix. In the U×I matrix, a horizontal line (row), U, indicates the number of users, and a vertical line (column), I, indicates the number of contents. The preference, r_ui, may be expressed as a number between 0 and 1. For example, as a value of r_ui is closer to 1, it may represent that the user's preference for a corresponding content is high. An entry which is not expressed with a number in the R matrix indicates that the user has not yet reacted to (or evaluated) the corresponding content. When a 5×5 matrix is R, R may be decomposed into P having a size of 5×3 and Q having a size of 3×5. If a matrix obtained by P×Q is R′, then P and Q for minimization of |R-R′| may be obtained by an optimization technique (e.g., gradient decent or alternating least square (ALS)). In this procedure, preference for a content that is not evaluated by the user may be predicted.

The electronic device may select at least one content expected to be preferred by the user from among contents and may recommend the selected content to the user, based on a result (the preference for the content not rated by the user) obtained by the prediction method as described above.

A content recommended to a user according to the prediction method may be considered to be recommended based on the relationship between contents evaluated by the user himself/herself and contents evaluated by another person. However, a content may be recommended to a user without explaining a specific reason for the recommendation.

The above information is presented as background information only to assist with an understanding of the disclosure. No determination has been made, and no assertion is made, as to whether any of the above might be applicable as prior art with regard to the disclosure.

### SUMMARY

Embodiments of the disclosure provide a method and apparatus that, when recommending a content, may increase the effect of recommendation by explaining a reason for the recommendation to a user.

According to an example embodiment, an electronic device may include: a communication circuit configured to communicate with an external electronic device; a memory configured to store user reaction data indicating reactions to contents based on users and biostatistic data for users; and a processor connected to the communication circuit and the memory, wherein the processor is configured to: calculate preference values for contents to which users have reacted using user reaction data; predict preference values for contents which have not been exposed to users, for the users using the calculated preference values; generate profiles related to states or activities for users using the biostatistic data; calculate reliability values for the generated profiles; calculate relevance values indicating degrees of relevance between profile types and contents based on the calculated preference values, the predicted preference values, and the calculated reliability values; select, from among contents which have not been exposed to a user selected from among the users, a content to be recommended to a selected user based on the predicted preference values; select, as a profile for generation of a recommendation reason, at least one of profiles of the selected user based on reliability values for the profiles of the selected user and relevance values between the recommended content and the profile types; and control the communication circuit to transmit the selected profile and the recommended content to an external electronic device of the selected user.

According to an example embodiments, and electronic device may include: a communication circuit configured to communicate with an external electronic device; a sensor configured to generate biometric data related to a state or activity of a user; a touch-sensitive display; a memory; and a processor connected to the communication circuit, the sensor, the display, and the memory, wherein the processor is configured to: collect, from the display, data indicating a reaction of the user to a content, and collect the biometric data from the sensor; control the communication circuit to transmit the user reaction data and the biometric data to the external electronic device; receive, from the external electronic device via the communication circuit, a user profile and a recommended content selected based on the data transmitted to the external electronic device; generate a recommendation reason for the recommended content using the user profile; and provide the user with the recommended content and the recommendation reason via the display.

According to various example embodiments, an electronic device can, while recommending a content to a user, explain a reason for recommending the content, thereby effectively indicating the necessity for the recommended content to the user and maximizing the effect of the recommendation.

In addition, various effects directly or indirectly identified via the disclosure may be provided.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a block diagram illustrating an example electronic device in a network environment according to various embodiments;

FIG. 2 is a block diagram illustrating an example system configured to support a content recommendation service according to various embodiments;

FIG. 3A is a diagram illustrating a first preference matrix generated in the system of FIG. 2 according to various embodiments;

FIG. 3B is a diagram illustrating a second preference matrix, a reliability matrix, and a relevance matrix generated in the system of FIG. 2 according to various embodiments;

FIG. 3C is a diagram illustrating an example of biostatistic data used for generating a user profile according to various embodiments;

FIG. 3D is a diagram for illustrating an example method of selecting a recommended content and a user profile using an alternative least square (ALS) algorithm in the system of FIG. 2 according to various embodiments;

FIG. 4A is a diagram illustrating an example of recommended content provided to a user along with a recommendation reason according to various embodiments;

FIG. 4B is a diagram illustrating an example recommended content provided to the user along with a recommendation reason according to various embodiments;

FIG. 5 is a flowchart illustrating example operations of a processor in a server according to various embodiments; and

FIG. 6 is a flowchart illustrating example operations of a processor in an electronic device according to various embodiments.

### DETAILED DESCRIPTION

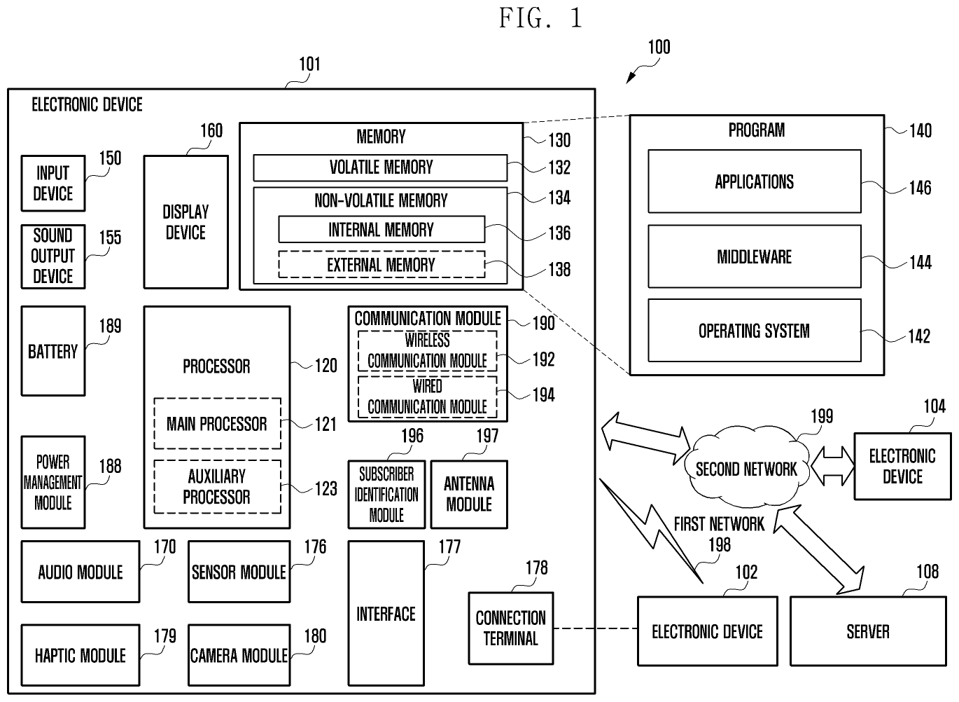

FIG. 1 is a block diagram illustrating an example electronic device 101 in a network environment 100 according to various embodiments. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor (e.g., an ECG (electrocardiogram) sensor, a PPG (photoplethysmography) sensor), a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to various embodiments may be one of various types of electronic devices. The electronic devices may include, for example, a portable communication device (e.g., a smartphone), a computer device, a portable multimedia device, a portable medical device, a camera, a wearable device, a home appliance, or the like. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), the element may be coupled with the other element directly (e.g., wiredly), wirelessly, or via a third element.

As used in connection with various embodiments of the disclosure, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

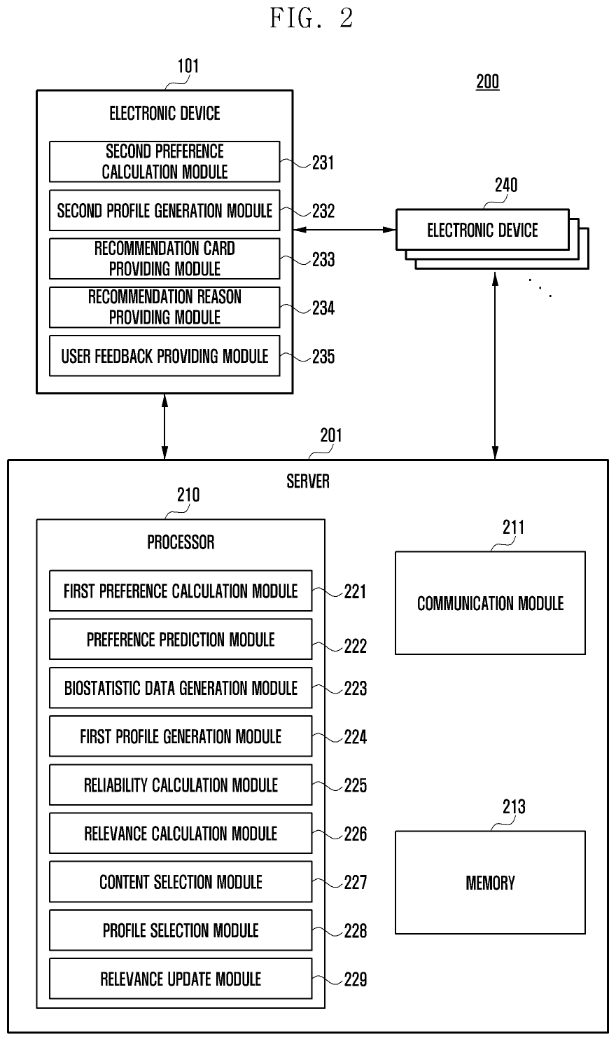

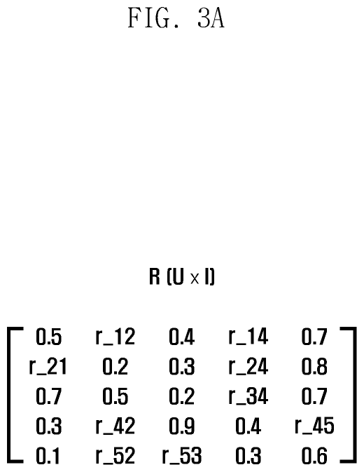

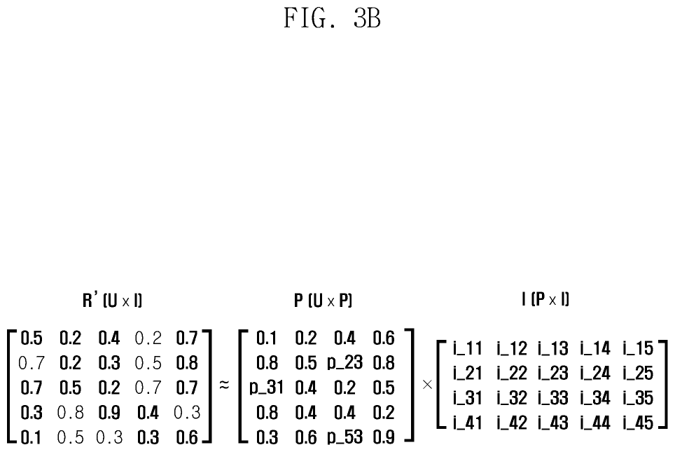

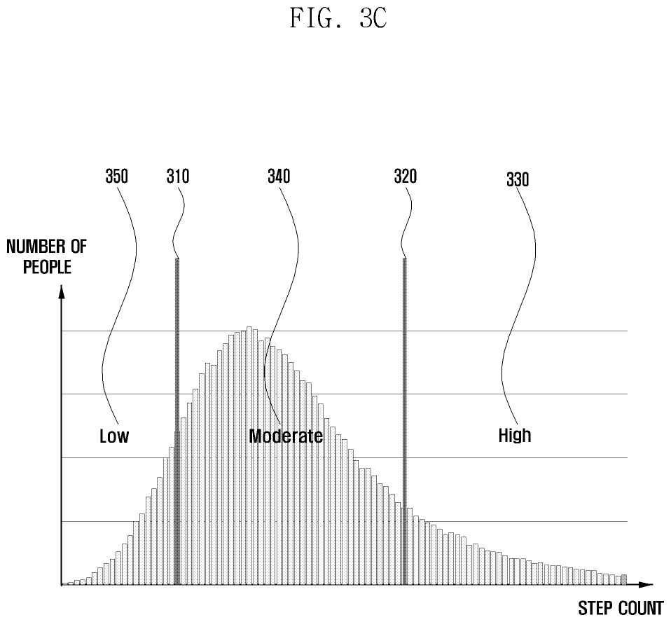

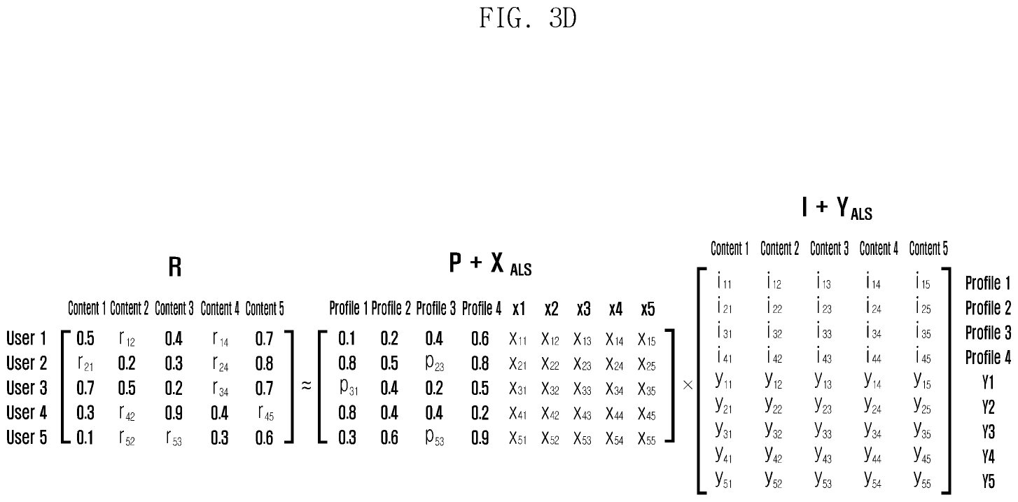

FIG. 2 is a block diagram illustrating an example system 200 configured to support a content recommendation service according to various embodiments. FIG. 3A is a diagram illustrating an example first preference matrix generated in the system 200 of FIG. 2 according to various embodiments. FIG. 3B is a diagram illustrating an example second preference matrix, a reliability matrix, and a relevance matrix generated in the system 200 of FIG. 2 according to various embodiments. FIG. 3C is a diagram illustrating an example of biostatistic data used for generating a user profile according to various embodiments. FIG. 3D is a diagram illustrating an example method of selecting a recommended content and a user profile using an alternative least square (ALS) algorithm in the system 200 of FIG. 2 according to various embodiments. In the description of FIG. 2 and FIG. 3, elements overlapping those of FIG. 1 may be omitted or briefly described.

Referring to FIG. 2, in the system 200, an electronic device (e.g., the electronic device 101 of FIG. 1) may communicate with a server 201 (e.g., the server 108 of FIG. 1) via a network (e.g., the first network 198 and/or the second network 199 of FIG. 1). The server 201 and electronic device 101 may also communicate with other electronic devices 240 (e.g., the electronic device 102 or 104 of FIG. 1) via the network.

According to an embodiment, the server 201 may include an element substantially the same as at least one of the elements of the electronic device 101. For example, the server 201 may include a communication module (e.g., including communication circuitry) 211, a processor (e.g., including processing circuitry) 210, and/or a memory 213. The processor 210 may include a first preference calculation module (e.g., including various processing circuitry and/or executable program elements) 221, a preference prediction module (e.g., including various processing circuitry and/or executable program elements) 222, a biostatistic data generation module (e.g., including various processing circuitry and/or executable program elements) 223, a first profile generation module (e.g., including various processing circuitry and/or executable program elements) 224, a reliability calculation module (e.g., including various processing circuitry and/or executable program elements) 225, and a relevance calculation module (e.g., including various processing circuitry and/or executable program elements) 226, a content selection module (e.g., including various processing circuitry and/or executable program elements) 227, a profile selection module (e.g., including various processing circuitry and/or executable program elements) 228, and/or a relevance update module (e.g., including various processing circuitry and/or executable program elements) 229. At least one of the modules 221, 222, 223, 224, 225, 226, 227, 228, or 229 may be included in the server 210, as separate hardware different from the processor 210. At least one of the modules 221, 222, 223, 224, 225, 226, 227, 228, or 229 may be software stored in the memory 213, and the processor 210 may execute the software.

According to an embodiment, the electronic device 101 may include a second preference calculation module (e.g., including various processing circuitry and/or executable program elements) 231, a second profile generation module (e.g., including various processing circuitry and/or executable program elements) 232, a recommendation card providing module (e.g., including various processing circuitry and/or executable program elements) 233, a recommendation reason providing module (e.g., including various processing circuitry and/or executable program elements) 234, and/or a user feedback providing module (e.g., including various processing circuitry and/or executable program elements) 235. At least one of the modules 231, 232, 233, 234, or 235 may be included in the processor 120 or may be included in the electronic device 101, as separate hardware different from the processor 120. At least one of the modules 231, 232, 233, 234, or 235 may be software stored in the memory 130, and the processor 120 may execute the software.

According to an embodiment, the electronic devices 240 may include the same element as at least one of the elements of the electronic device 101 of FIG. 1 or 2. For example, the electronic devices 240 may include an element that is substantially the same as at least one of the processor 120, the memory 130, the input module 150, the display module 160, the sensor module 176, the communication module 190, the second preference calculation module 231, the second profile generation module 232, the recommendation card providing module 233, the recommendation reason providing module 234, and/or the user feedback providing module 235.

According to an embodiment, the electronic devices 240 may be connected to the electronic device 101 and/or the server 201 via the communication module 190 (refer to FIG. 1). For example, some of the electronic devices 240 may be connected to the electronic device 101 via wired and/or wireless communication to transmit and/or receive data (e.g., a user's biometric data and/or data related to the user's activity). As another example, some of the electronic devices 240 may be connected to the server 201 via wired and/or wireless communication to transmit and/or receive data (e.g., a user's biometric data and/or data related to the user's activity).

According to an embodiment, the first preference calculation module 221 may include various processing circuitry and/or executable program elements and collect data indicating reactions of users to a specified content. For example, the electronic devices 101 or 240 may execute an application (e.g., a health application, a web browser, or a video streaming service program) to provide a user with a specified content or information (e.g., advertisement) related thereto via the display module 160. The electronic devices 101 or 240 may collect user reaction data for a content specified via the input module 150 or the display module 160, wherein the user reaction data may include raw data used for preference calculation, and includes, for example, a content viewing time, whether the content has been completely viewed, whether the content is purchased, whether “like” button is clicked, whether “dislike” button is clicked, or a satisfaction level of the user for the purchased product. The first preference calculation module 221 may receive user reaction data for the specified content, which is collected by each electronic device, from the electronic devices 101 or 240 via the communication module 211.

According to an embodiment, the first preference calculation module 221 may calculate a value indicating preference of a user for a content, to which the user has reacted, using collected user reaction data. In an embodiment, the first preference calculation module 221 may digitize the preference, r_ui, as, for example, a value between 0 and 1, based on user reaction data indicating a degree at which a user u likes a content i. For example, the first preference calculation module 221 may convert a preference value r_ui of the user u for the specified content i into a value closer to 1, as the user reaction data indicates a better reaction (or evaluation) of the user u for the specified content i.

According to an embodiment, the first preference calculation module 221 may generate a first preference matrix, R(U×I), having a horizontal line (row) corresponding to the number U of users (1, 2, 3, . . . , or U) and a vertical line (column) corresponding to the number I of contents (1, 2, 3, . . . , or I), and the calculated preference value of the user u for the specified content i may be recorded in a corresponding entry r_ui of R(U×I). According to an embodiment, notwithstanding the calculation of the preference value, an entry, the numerical value of which is not recorded, may be present in R(U×I). For example, in R(U×I) of FIG. 3A, r_12, r_14, r_21, r_24, r_34, r_42, r_45, r_52, and r_53 may correspond to entries with no record. Having no record of a preference value for a specific entry in R(U×I) may refer to the user not having yet reacted to (or evaluated) the specified content or may refer to that the user has not yet experienced the specified content. For example, r_12 may refer to that a second content is not exposed to a first user and thus has not yet been digitized.

According to an embodiment, the preference prediction module 222 may include various processing circuitry and/or executable program elements and predict a user's preference for a content, to which the user has not yet reacted, or a content that has not yet been exposed to the user, using a preference value calculated by the first preference calculation module 221. In an embodiment, the preference prediction module 222 may predict values of the entries with no record in R(U×I) using surrounding preference values. For example, the preference prediction module 222 may apply a prediction technique (e.g., matrix factorization) to R(U×I) having entries with no record, thereby generating a second preference matrix, R′(U×I), in which at least some of the entries with no record have preference values.

According to an embodiment, the biostatistic data generation module 223 may include various processing circuitry and/or executable program elements and generate biostatistic data using biometric data collected from the electronic devices 101 or 240. For example, the electronic devices 101 or 240 may collect biometric data related to a state or activity of a living body from an application (e.g., health application) via the sensor module 176 (a biometric sensor, a gyro sensor, or an accelerometer sensor) and/or the input module 150 or 160 (e.g., touch screen), wherein the biometric data may include raw data of biostatistic data and may include, for example, and without limitation, step count, active time, stress, heart rate, blood pressure, electrocardiogram, bioelectrical impedance analysis (BIA), body temperature, weight, body water, female cycle information, exercise type and duration, sleep duration, time of falling asleep, number of meals, meal time, food nutrient information, calories consumed, or the like. For example, the electronic device 101 may collect the biometric data (e.g., blood pressure, heart rate, exercise duration, and/or exercise type) from the electronic devices 240 (e.g., a wearable watch, wearable earbuds, or smart glasses) connected via the communication module 190.

According to an embodiment, the biometric data generation module 223 may receive biometric data collected by each electronic device from the electronic devices 101 or 240 via the communication module 211. The biostatistic data generation module 223 may process (or refine) the received biometric data using, for example, a statistical operator, thereby generating biostatistic data. For example, if the biometric data represents a total step count of the user u for a certain period (e.g., one week), the biometric data generation module 223 may average the total step count so as to obtain a daily step count of the user u. The biostatistic data generation module 223 may generate step count statistical data having a normal distribution characteristic as shown in the example of FIG. 3C, by classifying (e.g., classification into a 100 step unit) users into several groups on the basis of the daily step count for each user. In FIG. 3C, the X axis may represent the number of daily steps, and the Y axis may represent the number of people (users). According to an embodiment, the number of people and their average daily step counts may be known via the step count statistical data of FIG. 3C.

According to an embodiment, the first profile generation module 224 may include various processing circuitry and/or executable program elements and generate a user's profile using biometric data. In an embodiment, the first profile generation module 224 may identify, from the biostatistic data, an absolute position (e.g., level) of the user in a group according to criteria specified for each profile type, and may determine the identified position as the user's profile for the corresponding type. Referring to FIG. 3C, if the profile type is a daily step count, the first profile generation module 224 may determine the daily step count of the user to be one of a high level 330, a moderate level 340, or a low level 350 based on a first threshold 310 and a second threshold 320. The first profile generation module 224 may generate a step count profile indicating that the daily step count of the user is the determined level. In an embodiment, the first profile generation module 224 may compare a state (or activity) of the user with states (or activities) of other people, so as to identify, from the biostatistic data, a relative position (e.g., rank or percentage (%)) of the user in the group, thereby determining the identified position as the user's profile. The step count is one example of the profile types, and additional profile types that can be generated may include, for example, and without limitation, bedtime regularity, sleep duration, sleeplessness, sleep efficiency, sleep satisfaction, daily active time, daily exercise duration, exercise duration for each type (e.g., walking, running, cycling, using treadmill, or swimming), stress level, stress level regularity, daily calorie intake, frequency of excess sodium intake, mealtime regularity, daily resting heart rate, resting heart rate regularity, or the like.

According to an embodiment, the reliability calculation module 225 may include various processing circuitry and/or executable program elements and calculate a reliability value indicating to which extent a profile generated by the first profile generation module 224 is reliable as an actual profile of a user. For example, if a step count profile is generated based on 14 days, the reliability calculation module 225 may set a step count profile of a person to have a highest reliability if the person has daily step count data for all 14 days. According to distribution of step count data for 14 days, if step count data for at least 5 days is required to calculate a reliability, the reliability calculation module 225 may set a step count profile of a person to have a lowest reliability if the person has daily step count data for 5 days. As another example, the reliability calculation module 225 may set a step count profile of a person to have a low reliability if a daily step count of the person is too high (e.g., 1 million counts or more per day) or is too low (e.g., 10 counts or less). As another example, the reliability calculation module 225 may set a reliability value to be high if a step count level is maintained unchanged for a specified period (e.g., one month). The reliability calculation module 225 may set a reliability value to be lower as the range of a change increases. If a user of a step count profile with a high level 330 inevitably walks less than usual due to the influence of weather (e.g., heavy rain) during a specified period, and the step count profile is thus set to a lower level 350, a reliability of the corresponding profile may be set to be low.

According to an embodiment, the reliability calculation module 225 may generate a reliability matrix P(U×P) having a horizontal line (row) corresponding to the number U of users (1, 2, 3, . . . , or U) and a vertical line (column) corresponding to the number P of profile types (1, 2, 3, . . . , or P) that can be generated, and the calculated reliability value may be recorded in a corresponding entry (e.g., p_up) of P(U×P). Referring to FIG. 3B, there is no numerical value in entries p_23, P_31, and p_53, and this may be because there is no user profile for corresponding types with respect to designated users.

According to an embodiment, the relevance calculation module 226 may include various processing circuitry and/or executable program elements and calculate a relevance value indicating to what extent types (e.g., step count, obesity, or sleep quality) of user profiles generated by the first profile generation module 224 are related to a content, on the basis of a preference value (e.g., r_ui) for the content and reliability values (e.g., p_up) for the user profiles. In an embodiment, the relevance calculation module 226 may obtain a value i_pi indicating relevance between a profile type p (1, 2, 3, . . . , or P) and a content i (1, 2, 3, . . . , or I) using the matrix calculation equation “R′(U×I)≈P(U×P)×I(P×I)” of FIG. 3B.

According to an embodiment, the content selection module 227 may include various processing circuitry and/or executable program elements and select a content to be recommended to a user from among contents (e.g., unexposed contents) based on a preference value for each content. For example, referring to R(U×I) of FIG. 3A and R′(U×I) of FIG. 3B, a content that is not exposed to user #4 may be content #2 and content #5 corresponding to entries (e.g., r_42 and r_45) with no numerical value. In an embodiment, the content selection module 227 may select one of content #2 and content #5 as a recommended content.

According to an embodiment, the content selection module 227 may select, as the recommended content, a content predicted to be most preferred by the user, on the basis of the preference value for each content. For example, referring to R(U×I) of FIG. 3A and R′(U×I) of FIG. 3B, r_42 may be predicted to be 0.8 and r_45 may be predicted to be 0.3. The content selection module 227 may select, as the recommended content, content #2 corresponding to r_42 which has a high numerical value. The content selection module 227 may transmit the selected recommended content and/or information (e.g., a highlight video if the recommended content is a video) related thereto to an electronic device (e.g., the electronic device 101) of the user via the communication module 211.

According to an embodiment, the profile selection module 228 may include various processing circuitry and/or executable program elements and select at least one of the user profiles, as a profile that is reliable and relevant to the recommended content, on the basis of reliability values (e.g., p_up) for the user profiles and values (e.g., i_pi) indicating relevance between the content and the profile types. In an embodiment, the profile selection module 228 may perform an arithmetic operation (e.g., element-wise multiplication) on the reliability matrix P(U×P) and the relevance matrix I(P×I). For example, referring to FIG. 3B, if the recommended content is content #4 among the contents, and a user to be provided with the recommended content is user #4 among the users, the profile selection module 228 may perform the following arithmetic operation.

[0.8 0.4 0.4 0.2] ∘ [i_14 i_24 i_34 i_44] (=0.8*i_14, 0.4*i_24, 0.4*i_34, 0.2*i_44)

According to an embodiment, the profile selection module 228 may select a profile (e.g., a profile of a first type (e.g., step count, sleep quality, or obesity) corresponding to a highest value (0.8*i_14)), which corresponds to a highest value among result values of the operation, as a profile that is most reliable and most relevant to the recommended content (e.g., content #4). The profile selection module 228 may transmit the selected user profile to the user's electronic device (e.g., the electronic device 101) via the communication module 211.

According to an embodiment, the processor 210 may select a recommended content using the alternative least square (ALS) algorithm, and may select at least one of user profiles, as a profile that is reliable and relevant to the selected recommended content. For example, referring to FIG. 3D, the processor 210 may obtain values of respective entries from matrix R that is a first preference matrix, matrix P that is a reliability matrix, and matrix I that is a relevance matrix using a matrix factorization method. The processor 210 may select a recommended content using matrix R that is the first preference matrix. The processor 210 may select a user profile using matrix P that is the reliability matrix, and matrix I that is the relevance matrix. For example, the arithmetic operation described above may be used for selection of a user profile. Each entry (x_uf) of matrix X added as a vertical line (column) to matrix P in FIG. 3D may be a value indicating a relationship between a user u and a latent factor f. Each entry (y_fi) of matrix Y added as a horizontal row (row) to matrix I matrix may be a value indicating a relationship between a latent factor f and a content i. The processor 210 may determine the number of latent factors f to be a number that makes the product of matrix P+X and matrix I+Y to be U×I of matrix R. For example, if matrix R is 5×5 as illustrated, the number of f may be determined to be 5 so that matrix P+X is 5×9 and matrix I+Y is 9×5. As an algorithm for optimizing each entry (x_uf) of matrix X and each entry (y_fi) of matrix Y to have a smallest value, for example, gradient decent or alternating least square (ALS) may be used. ALS may be used when there are many users and/or contents, or when distributed processing is required. In order to select a recommended content and a user profile at the same time, it may be appropriate to use, as described above, a scheme of calculating entries of respective matrices using an algorithm, such as gradient decent or ALS, in a latent factor matrix configuration method in which matrix P is added to matrix X, and matrix I is added to matrix Y.

According to an embodiment, the relevance update module 229 may include various processing circuitry and/or executable program elements and receive a user's negative or positive feedback on a recommended content from an electronic device (e.g., the electronic device 101), and may update a relevance value using the received feedback. For example, the relevance update module 229 may update a relevance value using Equation 1 below.

current relevance value+(a−b)/number of users [Equation 1]

In Equation 1, a may refer to the number of positive feedback from users during one day, and b may refer to the number of negative feedback from users during one day.

Following the previous profile selection-related example, the relevance update module 229 may adjust, for example, relevance i_14 for profile type 1 and recommended content 4 on the basis of the received feedback. For example, in a case of negative feedback, the relevance update module 229 may adjust a relevance value to be one step (e.g., 0.1) lower. The relevance update module 229 may adjust the relevance value to a minimum value in response to reception of the negative feedback, thereby causing, when a profile is selected later, another profile to be selected. In a case of positive feedback, the relevance value may be adjusted to be one step higher.

According to an embodiment, the second preference calculation module 231 may include various processing circuitry and/or executable program elements and perform substantially the same function as that performed by the first preference calculation module 221 of the server 201. For example, the second preference calculation module 231 may collect data indicating reactions of a user of the electronic device 101 to a content, via the input module 150 or the display module 160, and may calculate a value (e.g., r_ui) indicating the user's preference for the content using the collected user reaction data. The second preference calculation module 231 may transmit the calculated preference value to the preference prediction module 222 of the server 201 via the communication module 190, so as to enable prediction of a preference for a content that has not been exposed to the user.

According to an embodiment, the second profile generation module 232 may include various processing circuitry and/or executable program elements and perform substantially the same function as that performed by the first profile generation module 224 of the server 201. In an embodiment, the second profile generation module 232 may identify, from biometric data collected by the electronic device 101, an absolute position (e.g., level) of the user in a group based on specified criteria, and may determine the identified position to be a user profile. For example, the second profile generation module 232 may recognize a daily step count of the user from the biometric data, wherein the second profile generation module 232 may determine a step count profile to be a high level if the recognized daily step count is 10,000 steps or more, may determine the step count profile to be a middle level if the recognized daily step count is 5,000 steps or more, and may determine the step count profile to be a low level if the recognized daily step count is less than 5,000 steps. The second profile generation module 232 may transmit the generated profile to the reliability calculation module 225 of the server 201 via the communication module 190, so as to enable calculation of a reliability value for the generated profile.

According to an embodiment, the recommendation card providing module 233 may include various processing circuitry and/or executable program elements and provide recommendation card templates to the recommendation reason providing module 234, to enable the recommendation reason providing module 234 to generate a recommendation reason.

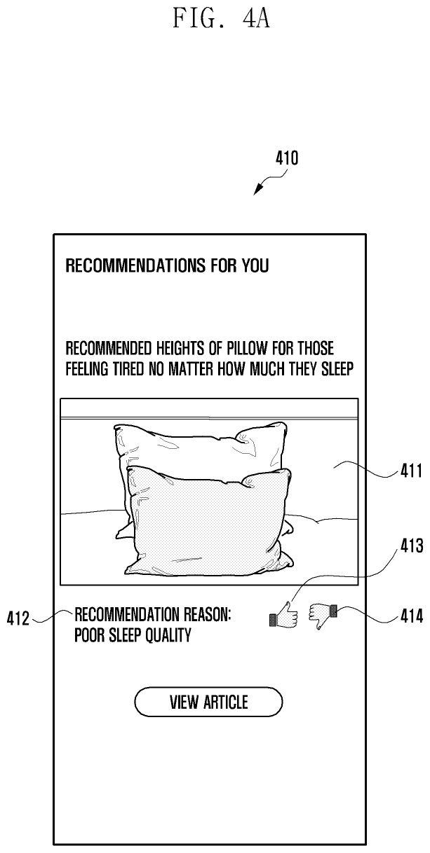

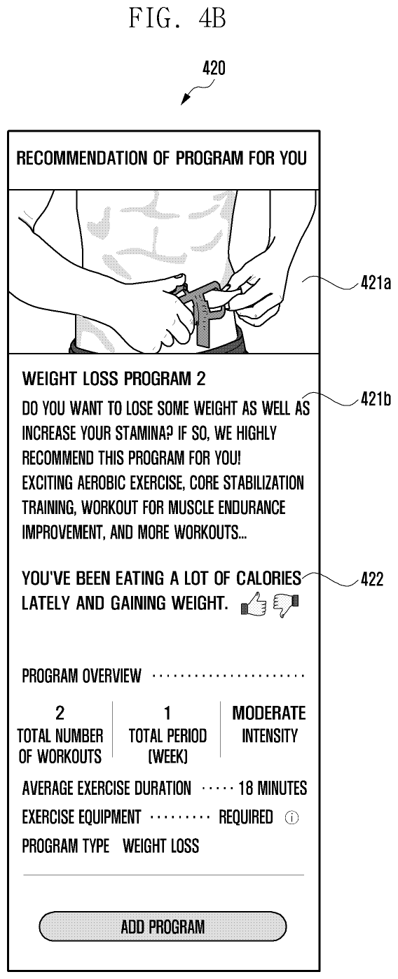

According to an embodiment, the recommendation card templates may include at least one graphic user interface (GUI) capable of generating a recommended content and a recommendation reason for recommending the content. For example, the recommendation card templates may include a template in the form of a designated card including at least one first GUI object (e.g., image, video or URL) for generating a recommended content, a second GUI object for generating a recommendation reason, or a third GUI object for collecting user feedback on the recommended content. Descriptions of a recommendation card will be provided and described in greater detail below with reference to FIG. 4A and FIG. 4B.

According to an embodiment, the recommendation reason providing module 234 may receive the recommended content and the user profile from the server 201 via the communication module 190. The recommendation reason providing module 234 may generate a recommendation reason for the recommended content using the received user profile. In an embodiment, the recommendation reason providing module 234 may select a recommendation card corresponding to the received user profile from among the recommendation card templates. For example, if a received sleep quality profile is of a low level, a recommendation card corresponding thereto may be selected as the recommendation card, wherein the corresponding recommendation card includes a phrase for a recommendation reason that is, for example, “a poor sleep quality”. As another example, if a received weight/calorie intake profile is of an overweight/overeating level, a recommendation card corresponding thereto may be selected as the recommendation card, wherein the corresponding recommendation card includes a phrase for a recommendation reason that is “You've been eating a lot of calories lately and gaining weight”. The recommendation reason providing module 234 may provide the selected recommendation card including the recommended content (or related information) along with the recommendation reason to the user via the display module 160.

According to an embodiment, the user feedback providing module 235 may include various processing circuitry and/or executable program elements and receive, via the display module 160, the user's feedback on the recommended content provided to the user, and may provide the feedback to the server 201 (e.g., the relevance update module 229) via the communication module 190 so as to update a relevance.

According to an embodiment, the communication module 211 may include various communication circuitry and support communication between the server 201 and each electronic device 101 or 240 using wired communication or wireless communication (e.g., Bluetooth (BT), Bluetooth low energy (BLE), or Wi-Fi).

According to an embodiment, the memory 213 may store various data used by at least one element (e.g., the processor 210 or the communication module 213) of the server 201. For example, the memory 213 may store the user's reaction data for the content collected from each electronic device 101 or 240.

FIG. 4A is a diagram illustrating an example recommended content provided to a user along with a recommendation reason according to various embodiments, and FIG. 4B is a diagram illustrating an example recommended content provided to the user along with a recommendation reason according to various embodiments. Referring to FIG. 4A, the processor 120 may add, to a recommendation card 410, a reason 412 for recommending a pillow, generated based on a profile received from the server 201, along with an image 411 representing the pillow, and may display the image and reason as a recommended content on the display module 160. Additionally, the processor 120 may add, to the recommendation card 410, buttons 413 or 414 for receiving a user's feedback on the pillow recommendation, to display the buttons on the display module 160. Referring to FIG. 4B, the processor 120 may add, to a recommendation card 420, a reason 422 for recommending a weight loss program, which is generated based on a profile received from the server 201, along with exercise program information 421b and an image 421a introducing the weight loss program, and may display the program, image, and reason as a recommended content on the display module 160.

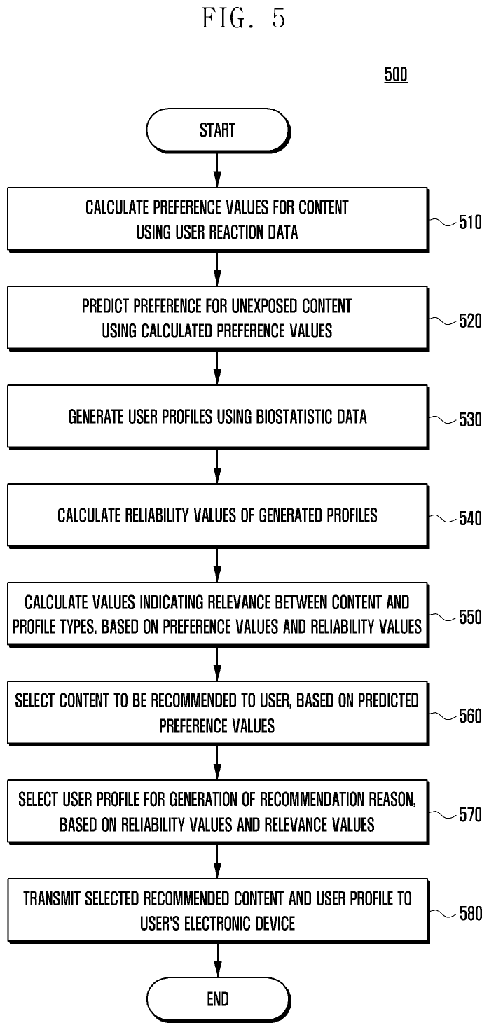

FIG. 5 is a flowchart 500 illustrating example operations of the processor 210 in the server 201 according to various embodiments. The operations may include operations 510, 520, 530, 540, 550, 560, 570 and 580 (which may hereinafter be referred to as operations 510 to 580). According to an embodiment, at least one of operations 510 to 580 may be omitted, the order of some operations may be changed, or other operations may be added. The processor 210 may perform operations 510 to 580 using at least one of the modules 221, 222, 223, 224, 225, 226, 227, 228, or 229.

In operation 510, the processor 210 may calculate values indicating preferences of respective users for contents using data indicating reactions of the users (1, 2, 3, . . . or U) to the contents, which are collected by the electronic devices 101 or 240. The processor 210 may convert, into values between 0 and 1, the reactions of the users to the contents. For example, the processor 210 may convert a preference value to a value close to 0 when the user's reaction is negative, and may convert a preference value to a value close to 1 when the user's reaction is positive.

In operation 520, using the preference values calculated for the respective users for the contents, the processor 210 may predict preferences of the respective users for contents (hereinafter, unexposed contents) which have not been exposed to the users. The unexposed contents among contents (1, 2, 3, . . . , or I) which can be provided to the users may be different for each user, and the aforementioned matrix factorization may be used as a prediction technique, for example.

In operation 530, the processor 210 may generate profiles (e.g., step count rank, sleep quality, or obesity) related to states or activities of the respective users using biostatistic data generated using raw data collected by the electronic devices 101 or 240. The collected raw data may be different for each user, and the generated profiles may be different for each user. For example, raw data related to an obesity profile may be collected from an electronic device of a first user to the server 201, but not from an electronic device of a second user to the server 201. Accordingly, only an obesity profile of the first user may be generated.

In operation 540, the processor 210 may compare the generated profiles with existing profiles of the same types, thereby calculating values indicating reliabilities of the profiles generated for the respective users. In an embodiment, the processor 210 may identify, from the biostatistic data, absolute positions of the users in a group according to criteria specified for each profile type, and may determine the identified positions as profiles of the corresponding type. The processor 210 may calculate reliability values of the corresponding profiles on the basis of variations of the positions during a specified period. For example, the processor 210 may set a reliability value of a step count profile in proportion to the number of days, for which daily step count data exists, during a specified period (e.g., 14 days). For example, a reliability of a person having daily step count data for all 14 days may be set to be highest, a reliability of a person having daily step count data for 5 days out of 14 days may be set to be lowest, and a reliability of a person having daily step count data of less than 5 days may not be set. As another example, the processor 210 may set a reliability of a step count profile of a person to be low if a daily step count of the person is too high (e.g., 1 million counts or more per day) or is too low (e.g., 10 counts or less). As another example, the processor 210 may set a reliability value to be high if a step count level is maintained unchanged for a specified period (e.g., one month). The processor 210 may set a reliability value to be lower as the range of a change increases.

In operation 550, the processor 210 may calculate relevance values indicating to what extent the profile types are related to contents, based on the preference values calculated (or predicted) for each content for the respective users and the reliability values for the profiles generated for the respective users. In an embodiment, the processor 210 may obtain a relevance value i_pi indicating relevance between profile type p and content i using the matrix calculation equation as shown in FIG. 3C.

In operation 560, the processor 210 may select a content to be recommended to a user u (e.g., a user of the electronic device 101, who has requested a recommended content via execution of a specified application (e.g., health application)) selected from the users, from among contents which have not been exposed to the user u, based on the preference value predicted for each content.

In operation 570, the processor 210 may select at least one of the profiles of the user as a profile for generation of a recommendation reason, based on the reliability values for the profiles of the user and the relevance values between the recommended content and the profile types.

In operation 580, the processor 210 may transmit the selected profile and the recommended content to the electronic device of the user via the communication module 211.

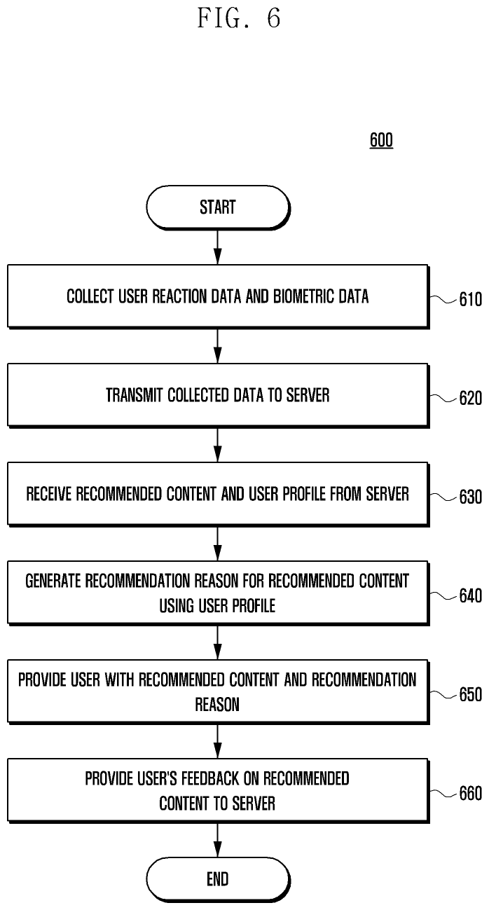

FIG. 6 is a flowchart 600 illustrating example operations of the processor 120 in the electronic device 101 according to various embodiments. The operations may include operations 610, 620, 630, 640, 650 and 660 (which may hereinafter be referred to as operations 610 to 660). According to an embodiment, at least one of operations 610 to 660 may be omitted, the order of some operations may be changed, or other operations may be added. The processor 120 may perform operations 610 to 660 using at least one of the modules 231, 232, 233, 234, or 235.

In operation 610, the processor 120 may collect data indicating a user's reaction to a content via an input device (e.g., touchscreen), and may collect biometric data related to a state or activity of the user via the sensor module 176 (e.g., a biometric sensor, a gyro sensor, or an accelerometer sensor) or the input device. In an embodiment, the processor 120 may collect data indicating a user's reaction to a content and/or biometric data related to a state or activity of the user from some of electronic devices (e.g., the electronic devices 240 of FIG. 2) connected via the communication module 190.

In operation 620, the processor 120 may transmit the collected data (user reaction data and/or biometric data) to the server 201 via the communication module 190.

In operation 630, the processor 120 may receive a user profile and a recommended content (and/or related information) selected based on the data transmitted to the server 201, from the server 201 via the communication module 190.

In operation 640, the processor 120 may generate a recommendation reason for the recommended content using the user profile received from the server 201. For example, the processor 120 may select a recommendation card including a recommendation reason corresponding to the received user profile from among recommendation card templates stored in the memory 130.

In operation 650, the processor 120 may provide the recommended content (and/or related information) and the recommendation reason, which are included in, for example, the recommendation card, to the user via the display module 160.

In operation 660, the processor 120 may receive, from the display module 160, the user's feedback on the recommended content provided to the user, and may provide the feedback to the server 201 via the communication module 190 so as to enable a relevance to be updated.

An electronic device (e.g., the server 201) in various example embodiments may include: a communication circuit configured to communicate with an external electronic device; a memory configured to store user reaction data indicating reactions to contents and biostatistic data for users; and a processor connected to the communication circuit and the memory, wherein the processor is configured to: calculate preference values for contents, to which users have reacted for users using the user reaction data; predict preference values for contents which have not been exposed to users for users using the calculated preference values; generate profiles related to states or activities for users using the biostatistic data; calculate reliability values for the generated profiles; calculate relevance values indicating degrees of relevance between profile types and contents based on the calculated preference values, the predicted preference values, and the calculated reliability values; select, from among contents which have not been exposed to a user selected from among the users, a content to be recommended to the selected user based on the predicted preference values; select, as a profile for generation of a recommendation reason, at least one of profiles of the selected user based on reliability values for the profiles of the selected user and relevance values between the recommended content and the profile types; and control the communication circuit to transmit the selected profile and the recommended content to an external electronic device of the selected user.

The processor may be configured to, as a part of operations of generating the profiles, identify, from the biostatistic data, absolute positions of the users in a group based on criteria specified for each profile type, and may determine the identified positions as profiles of the corresponding type. The processor may be configured to, as a part of operations of calculating the reliability values, calculate the reliability values based on variations of the positions during a specified period. The processor may be configured to set the reliability values to be lower as the variations increase.

The processor may be configured to perform, as a part of operations of calculating the preference values, recording a preference value for a content i, to which a user u has reacted, in a corresponding entry r_ui of a first preference matrix R(U×I) having a row corresponding to the number U of users and a column corresponding to the number I of contents; as a part of operations of predicting the preference values, generating, by applying matrix factorization to the first preference matrix including entries with no preference value recorded therein, a second preference matrix R′(U×I) in which a preference value is recorded in at least a part of the entries with no record; as a part of operations of calculating the reliability values, recording a reliability value, which is calculated for a profile type p generated in accordance with the user u, in a corresponding entry p up of a reliability matrix P(U×P) having a row corresponding to the number U of users and a column corresponding to the number P of profile types; and as a part of operations of calculating the relevance values, calculating a value i_pi indicating relevance between the profile type p and the content i, using the equation below.

The processor may be configured to, as a part of operations of selecting the profile, based on the selected user being an Nth user and the recommended content being an Mth content, perform an arithmetic operation (element-wise) on an Nth row in the reliability matrix P(U×P) and an Mth column in the relevance matrix I(P×I); and select a profile corresponding to a largest value among values obtained as a result of the arithmetic operation, as a profile for generation of a recommendation reason.

The processor may be configured to receive feedback on the recommended content from an external electronic device of the selected user, and to update a relevance value of an entry corresponding to a largest value in the relevance matrix based on the received feedback.

The processor may be configured to, based on the received feedback being negative feedback, adjust the relevance value of the entry corresponding to the largest value to be low; and based on the received feedback being positive feedback, adjust the relevance value of the entry corresponding to the largest value to be high.

An electronic device (e.g., the electronic device 101) in various example embodiments may include: a communication circuit configured to communicate with an external electronic device; a sensor configured to generate biometric data related to a state or activity of a user; a touch-sensitive display; a memory; and a processor connected to the communication circuit, the sensor, the display, and the memory, wherein the processor is configured to: collect, from the display, data indicating a reaction of the user to a content, and collect the biometric data from the sensor; control the communication circuit to transmit the user reaction data and the biometric data to the external electronic device; receive, from the external electronic device via the communication circuit, a user profile and a recommended content selected based on the data transmitted to the external electronic device; generate a recommendation reason for the recommended content using the user profile; and provide the user with the recommended content and the recommendation reason via the display.

The processor may be configured to receive the user's feedback on the recommended content from the display, and to provide the feedback to the external electronic device via the communication circuit.

The processor may be configured to select a recommendation card including a recommendation reason corresponding to the user profile from among recommendation card templates stored in the memory.

While the disclosure has been illustrated and described with reference to various example embodiments, it will be understood that the various example embodiments are intended to be illustrative, not limiting. It will be further understood by those skilled in the art that various changes in form and detail may be made without departing from the true spirit and full scope of the disclosure, including the appended claims and their equivalents.

What is claimed is:

1. An electronic device comprising:
a communication circuit configured to communicate with an external electronic device;
a memory configured to store user reaction data indicating reactions to contents for users and biostatistic data for users; and
a processor connected to the communication circuit and the memory, wherein the processor is configured to:
calculate preference values for contents, to which users have reacted, for users using the user reaction data;
predict preference values for contents, which have not been exposed to the users, for users using the calculated preference values;
generate profiles related to states or activities for users using the biostatistic data;
calculate reliability values for the generated profiles;
calculate relevance values indicating degrees of relevance between profile types and contents based on the calculated preference values, the predicted preference values, and the calculated reliability values;
select, from among contents which have not been exposed to a user selected from among the users, a content to be recommended to the selected user, based on the predicted preference values;
select, as a profile for generation of a recommendation reason, at least one of profiles of the selected user based on reliability values for the profiles of the selected user and relevance values between the recommended content and the profile types; and
control the communication circuit to transmit the selected profile and the recommended content to an external electronic device of the selected user.

2. The electronic device of claim 1, wherein the profile types comprise at least one of: daily step count, obesity, sleep quality, sleep duration, sleep efficiency, sleeplessness, bedtime regularity, daily exercise duration, daily calorie intake, mealtime, stress level, and daily heart rate at rest.

3. The electronic device of claim 1, wherein the processor is configured, as a part of the generating of the profiles, to:
identify, from the biostatistic data, absolute positions of users in a group according to criteria specified for each profile type; and
determine the identified positions as profiles of a corresponding type.

4. The electronic device of claim 3, wherein the processor is configured, as a part of the calculating of the reliability values, to calculate the reliability values based on variations of the positions during a specified period.

5. The electronic device of claim 4, wherein the processor is configured to set the reliability values to be lower as the variations increase.

6. The electronic device of claim 1, wherein the processor is configured to:
as a part of the calculating of the preference values, record a preference value for a content i, to which a user u has reacted, in a corresponding entry r_ui of a first preference matrix R(U×I) having a row corresponding to a number U of users and a column corresponding to a number I of contents;
as a part of the predicting of the preference values, generate, by applying matrix factorization to a first preference matrix comprising entries with no preference value recorded therein, a second preference matrix R′(U×I) in which a preference value is recorded in at least a part of the entries with no recorded preference value;
as a part of the calculating of the reliability values, record a reliability value, calculated for a profile type p generated for the user u, in a corresponding entry p_up of a reliability matrix P(U×P) having a row corresponding to the number U of users and a column corresponding to a number P of profile types; and
as a part of the calculating of the relevance values, calculate a value i_pi indicating relevance between the profile type p and the content i, using a matrix equation,
R′(U×I)≈P(U×P)×I(P×I),
where R′(U×I) is the second preference matrix, P(U×P) is the reliability matrix, and I(P×I) is a relevance matrix having a row corresponding to the number P of profile types and a column corresponding to the number I of contents.

7. The electronic device of claim 6, wherein the processor is configured, as a part of the selecting the profile, to:
based on the selected user being an Nth user and the recommended content being an Mth content, perform arithmetic operation on an Nth row in the reliability matrix P(U×P) and an Mth column in the relevance matrix I(P×I); and
select a profile corresponding to a largest value among values obtained as a result of the arithmetic operation, as a profile for generation of a recommendation reason.

8. The electronic device of claim 7, wherein the processor is configured to:
receive feedback on the recommended content from an external electronic device of the selected user; and
update a relevance value of an entry corresponding to the largest value in the relevance matrix based on the received feedback.

9. The electronic device of claim 8, wherein the processor is configured to:
based on the received feedback being negative feedback, adjust the relevance value of the entry corresponding to the largest value to be low; and
based on the received feedback being positive feedback, adjust the relevance value of the entry corresponding to the largest value to be high.

10. An electronic device comprising:
a communication circuit configured to communicate with an external electronic device;
a sensor configured to generate biometric data related to a state or activity of a user;
a touch-sensitive display;
a memory; and
a processor connected to the communication circuit, the sensor, the display, and the memory, wherein the processor is configured to:
collect, from the display, data indicating a reaction of the user to a content, and collect the biometric data from the sensor;
control the communication circuit to transmit the user reaction data and the biometric data to the external electronic device;
receive, from the external electronic device via the communication circuit, a user profile and a recommended content selected based on the data transmitted to the external electronic device;
generate a recommendation reason for the recommended content using the user profile; and
provide the user with the recommended content and the recommendation reason via the display.

11. The electronic device of claim 10, wherein the processor is configured to:
receive feedback of the user on the recommended content from the display; and
provide the feedback to the external electronic device via the communication circuit.

12. The electronic device of claim 10, wherein the processor is configured to select a recommendation card comprising a recommendation reason corresponding to the user profile from among recommendation card templates stored in the memory.

13. A method for operating an electronic device, the method comprising:
calculating preference values for contents, to which users have reacted, for users using user reaction data indicating reactions to the contents for users;
predicting preference values for contents, which have not been exposed to the users, for users using the calculated preference values;
generating profiles related to states or activities for users using biostatistic data for the users;
calculating reliability values for the generated profiles;
calculating relevance values indicating degrees of relevance between profile types and contents based on the calculated preference values, the predicted preference values, and the calculated reliability values;
selecting, from among contents which have not been exposed to a user selected from among the users, a content to be recommended to the selected user, based on the predicted preference values;
selecting, as a profile for generation of a recommendation reason, at least one of profiles of the selected user based on reliability values for the profiles of the selected user and relevance values between the recommended content and the profile types; and
transmitting the selected profile and the recommended content to an external electronic device of the selected user via a communication circuit of the electronic device.

14. The method of claim 13, wherein the generating of the profiles comprises:
identifying, from the biostatistic data, absolute positions of users in a group according to criteria specified for each profile type; and
determining the identified positions as profiles of a corresponding type.

15. The method of claim 14, wherein the calculating of the reliability values comprises calculating the reliability values based on variations of the positions during a specified period.

16. The method of claim 15, wherein the calculating of the reliability values comprises setting the reliability values to be lower as the variations increase.

17. The method of claim 13, wherein:
the calculating of the preference values comprises: recording a preference value for a content i, to which a user u has reacted, in a corresponding entry r_ui of a first preference matrix R(U×I) having a row corresponding to a number U of users and a column corresponding to a number I of contents;
the predicting of the preference values comprises generating, by applying matrix factorization to the first preference matrix comprising entries with no preference value recorded therein, a second preference matrix R′(U×I) in which a preference value is recorded in at least a part of the entries with no recorded preference value;
the calculating of the reliability values comprises recording a reliability value, calculated for a profile type p generated in for the user u, in a corresponding entry p up of a reliability matrix P(U×P) having a row corresponding to the number U of users and a column corresponding to the number P of profile types; and
the calculating of the relevance values comprises calculating a value i_pi indicating relevance between the profile type p and the content i, using a matrix equation,
R′(U×I)≈P(U×P)×I(P×I),
where R′(U×I) is the second preference matrix, P(U×P) is the reliability matrix, and I(P×I) is a relevance matrix having a row corresponding to the number P of profile types and a column corresponding to the number I of contents.

18. The method of claim 17, wherein the selecting of the profile comprises:
based on the selected user being an Nth user and the recommended content being an Mth content, performing arithmetic operation on an Nth row in the reliability matrix P(U×P) and an Mth column in the relevance matrix I(P×I); and
selecting a profile corresponding to a largest value among values obtained as a result of the arithmetic operation, as a profile for generation of a recommendation reason.

19. The method of claim 18, further comprising:
receiving feedback on the recommended content from an external electronic device of the selected user; and
updating a relevance value of an entry corresponding to the largest value in the relevance matrix based on the received feedback.

20. The method of claim 19, wherein the updating of the relevance value comprises:
based on the received feedback being negative feedback, adjusting the relevance value of the entry corresponding to the largest value to be low; and
based on the received feedback being positive feedback, adjusting the relevance value of the entry corresponding to the largest value to be high.
